(19) United States
(12) Patent Application Publication
(10) Pub. No.: US 2010/0130560 A1
(43) Pub. Date: May 27, 2010

(54) FUNGICIDE 2-PYRIDYL-METHYLENE-THIO CARBOXAMIDE OR 2-PYRIDYL-METHYLENE-N-SUBSTITUTED CARBOXIMIDAMIDE DERIVATIVES

(75) Inventors: Philippe Desbordes, Lyon (FR); Stephanie Gary, Champagne au Mont d'Or (FR); Marie-Claire Grosjean-Cournoyer, Curis-Au-Mont-d'Or (FR); Benoit Hartmann, Sainte-Foy-Les-Lyon (FR); Philippe Rinolfi, Chatillon D-Azergues (FR); Jean-Pierre Vors, Sainte Foy Les Lyon (FR)

Correspondence Address: OSTROLENK FABER GERB & SOFFEN, 1180 AVENUE OF THE AMERICAS, NEW YORK, NY, 100368403, US

(73) Assignee: Bayer SAS, Lyon (FR)

(21) Appl. No.: 12/452,893

(22) PCT Filed: Jul. 31, 2008
(86) PCT No.: PCT/EP2008/060038

§ 371 Date: Jan. 26, 2010

(30) Foreign Application Priority Data
Jul. 31, 2007 (EP) 07356106.0

(51) Int. Cl. A01N 43/56; C07D 401/12; A01P 3/00

(52) U.S. Cl. 514/341; 546/275.4

(57) ABSTRACT

The present invention relates 2-pyridyl-methylene-thiocarboxamide or 2-pyridyl-methylene-N-substituted-carboximidamide derivatives of formula (I) wherein A represents a carbo-linked 5-membered heterocyclyl group; T represents S, N—R^{a}, N—OR^{a}, N—NR^{a}R^{b} or N—CN; Z¹ represents a substituted or non substituted C₃-C₇ cycloalkyl; Y represents a C₁-C₅-halogenoalkyl comprising up to 9 halogen atoms that can be the same or different; Z², Z³, Y and n represent various substituents; their process of preparation; their use as fungicide active agents, particularly in the form of fungicide compositions and methods for the control of phytopathogenic fungi, notably of plants, using these compounds or compositions.

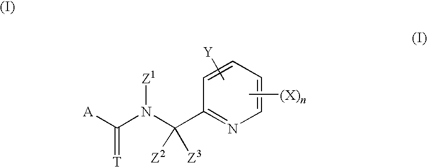

The present invention relates to 2-pyridyl-methylene-thiocarboxamide or 2-pyridyl-methylene-N-substituted-carboximidamide derivatives, their process of preparation, their use as fungicide active agents, particularly in the form of fungicide compositions, and methods for the control of phytopathogenic fungi, notably of plants, using these compounds or compositions.

In international patent application WO-01/11966 certain 2-pyridyl-methylene-carboxamide derivatives are generically embraced in a broad disclosure of numerous compounds.

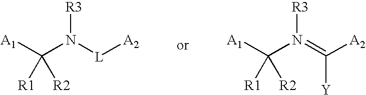

However, this document does not specifically disclose nor suggest to select such compounds wherein the nitrogen atom of the carboxamide residue might be substituted by a cycloalkyl.

In international patent application WO-96/38419 certain 2-pyridyl-methylene-thiocarboxamide derivatives are generically embraced in a broad disclosure of numerous compounds of the following formula:

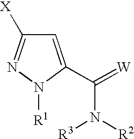

wherein X represents halogen, W can represent a sulphur atom, R¹ can represent C₁-C₄-alkyl, R² can represent C₃-C₇-cycloalkyl and R³ can represent various substituents among that heterocycles. However, this document does not specifically disclose nor suggest to select such compounds wherein the nitrogen atom of the thiocarboxamide residue can be substituted by a cycloalkyl.

In international patent application WO-06/098128 certain 2-pyridyl-methylene-thiocarboxamide derivatives are generically embraced in a broad disclosure of numerous compounds of the following formula:

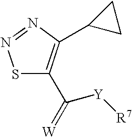

wherein W can represent a sulphur atom, Y can represent N-cycloalkyl and R⁷ can represent various substituents among that heterocycles. However, this document does not specifically disclose nor suggest to select such compounds wherein the nitrogen atom of the thiocarboxamide residue can be substituted by a cycloalkyl.

It is always of high-interest in agriculture to use novel pesticide compounds in order to avoid or to control the development of resistant strains to the active ingredients. It is also of high-interest to use novel compounds being more active than those already known, with the aim of decreasing the amounts of active compound to be used, whilst at the same time maintaining effectiveness at least equivalent to the already known compounds. We have now found a new family of compounds that possess the above mentioned effects or advantages.

Accordingly, the present invention provides 2-pyridyl-methylene-thiocarboxamide or 2-pyridyl-methylene-N-substituted-carboximidamide derivatives of formula (I)

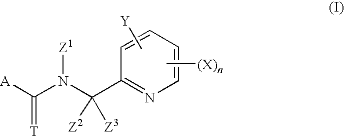

wherein
A represents a carbo-linked, substituted or non substituted, unsaturated or partially saturated, 5-membered heterocyclyl group;
T represents S, N—R^{a}, N—OR^{a}, N—NR^{a}R^{b} or N—CN;
Z¹ represents a substituted or non substituted C₃-C₇ cycloalkyl;
Z² and Z³, that can be the same or different, represent a hydrogen atom; C₁-C₅-alkyl; C₂-C₅-alkenyl; C₂-C₅-alkynyl; cyano; nitro; a halogen atom; C₁-C₅-alkoxy; C₂-C₅-alkenyloxy; C₂-C₅-alkynyloxy; C₃-C₇-cycloalkyl; C₁-C₅-alkylsulphenyl; an amino; C₁-C₅-alkylamino; di-C₁C₅-alkylamino; C₁-C₅-alkoxycarbonyl; C₁-C₅-alkylcarbamoyl; di-C₁-C₅-alkylcarbamoyl; N—C₁-C₅-alkyl-C₁-C₅-alkoxycarbamoyl; or
Z² and Z³ together with the carbon atom to that they are linked can form a 3-, 4-, 5- or 6-membered carbo-or heterocyclic ring, that can be substituted;
Y represents a C₁-C₅-halogenoalkyl comprising up to 9 halogen atoms that can be the same or different;
X, that can be the same or different, represents a halogen atom; nitro; cyano; hydroxyl; a carboxyl group; C₁-C₈-alkyl; C₁-C₆-halogenoalkyl comprising up to 9 halogen atoms that can be the same or different; C₁-C₈-alkylamino; di-C₁-C₈-alkylamino; C₁-C₈-alkoxy; C₁-C₆-halogenoalkoxy comprising up to 9 halogen atoms that can be the same or different; C₁-C₈-alkylsulfanyl; C₁-C₆-halogenoalkylsulfanyl comprising up to 9 halogen atoms that can be the same or different; C₂-C₈-alkenyloxy; C₂-C₈-halogenoalkenyloxy comprising up to 9 halogen atoms that can be the same or different; C₃-C₈-alkynyloxy; C₃-C₈-halogenoalkynyloxy comprising up to 9 halogen atoms that can be the same or different; C₃-C₈-cycloalkyl; C₁-C₈-alkoxycarbonyl; C₁-C₈-alkylsulphinyl; C₁-C₈-alkylsulphonyl; C₁-C₈-halogenoalkylsulphinyl comprising up to 9 halogen atoms that can be the same or different; C₁ C₈-halogenoalkylsulphonyl comprising up to 9 halogen atoms that can be the same or different or C₁-C₆-alkoximino-C₁-C₆-alkyl;
n represents 0, 1, 2 or 3;
R^{a} and R^{b}, that can be the same or different, represent a hydrogen atom; C₁-C₈-alkyl; C₁-C₈-halogenoalkyl comprising up to 9 halogen atoms that can be the same or different; C₁-C₈-alkoxy-C₁-C₈-alkyl; C₂-C₈-alkenyl; C₂-C₈-halogenoalkenyl comprising up to 9 halogen atoms that can be the same or different; C₂-C₈-alkynyl; C₂-C₈-halogenoalkynyl comprising up to 9 halogen atoms that can be the same or different; C₃-C₇-cycloalkyl; C₃-C₇-cycloalkyl-C₁-C₈-alkyl; C₃-C₇-halogenocycloalkyl comprising up to 9 halogen atoms that can be the same or different; formyl; C₁-C₈-alkylcarbonyl; C₁-C₈-halogenoalkylcarbonyl comprising up to 9 halogen atoms that can be the same or different; C₁-C₈-alkylsulphonyl; C₁-C₈-halogenoalkylsulphonyl comprising up to 9 halogen atoms that can be the same or different; phenyl that can be substituted by up to 5 groups Q; naphthyl that can be substituted by up to 6 groups Q; phenylmethylene that can be substituted by up to 5 groups Q; phenylsulphonyl that can be substituted by up to 5 groups Q;
Q, that can be the same or different, represents a halogen atom; cyano; nitro; C₁-C₈-alkyl; C₁-C₈-alkoxy; C₁-C₈-alkylsulfanyl; C₁-C₈-halogenoalkyl comprising up to 9 halogen atoms that can be the same or different; C₁-C₈-halogenoalkoxy comprising up to 9 halogen atoms that can be the same or different;
   as well as salts, N-oxides, metallic complexes, metalloidic complexes and optically active or geometric isomers thereof.

Any of the compounds according to the invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods that are known per se by the man ordinary skilled in the art.

Any of the compounds according to the invention can also exist in one or more geometric isomer forms depending on the number of double bonds in the compound. The invention thus relates equally to all geometric isomers and to all possible mixtures, in all proportions. The geometric isomers can be separated according to general methods that are known per se by the man ordinary skilled in the art.

For the compounds according to the invention, the following generic terms are generally used with the following meanings:
halogen means either one of fluorine, bromine, chlorine or iodine;
heteroatom can be nitrogen, oxygen or sulphur;
any alkyl group, alkenyl group or alkynyl group can be straight or branched;
in the case of an amino group or the amino moiety of any other amino-containing group, substituted by two substituents that can be the same or different, the two substituents together with the nitrogen to that they are attached can form a heterocyclyl group, preferably a 5 to 7-membered heterocyclyl group, that can be substituted and can contain other hetero atoms, for example morpholino or piperidinyl.

Preferred compounds of formula (I) according to the invention are those wherein A is selected in the list consisting of:
a heterocycle of formula (A¹)

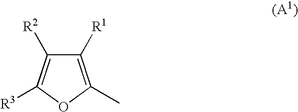

wherein R¹ to R³ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²)

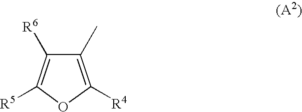

wherein R⁴ to R⁶ that can be the same or different represent a hydrogen atom; a halogen atom; C₁-C₅-alkyl or C₁-C₅-halogenoalkyl comprising up to 5 halogen atoms that can be the same or different;
a heterocycle of formula (A³)

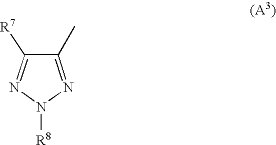

wherein R⁷ represents a hydrogen atom; a halogen atom; C₁-C₅-alkyl or C₁-C₅-halogenoalkyl comprising up to 5 halogen atoms that can be the same or different;
R⁸ represents a hydrogen atom; C₁-C₅-alkyl or a phenyl substituted by a halogen atom or by a C₁-C₅-alkyl;
a heterocycle of formula (A⁴)

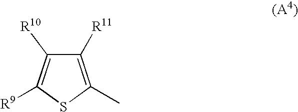

wherein R⁹ to R¹¹ that can be the same or different represent a hydrogen atom; a halogen atom; C₁-C₅-alkyl; an amino; C₁-C₅-alkoxy; C₁-C₅-alkylsulfanyl or C₁-C₅-halogenoalkyl comprising up to 5 halogen atoms that can be the same or different;
a heterocycle of formula (A⁵)

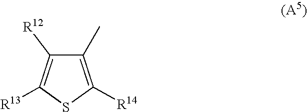

wherein

R¹² and R¹³ that can be the same or different represent a hydrogen atom; a halogen atom; C₁-C₅-alkyl; C₁-C₅-alkoxy; an amino or C₁-C₅-halogenoalkyl comprising up to 5 halogen atoms that can be the same or different;

R¹⁴ represents a hydrogen atom; a halogen atom; C₁-C₅-alkyl; an amino or C₁-C₅-halogenoalkyl comprising up to 5 halogen atoms that can be the same or different;
a heterocycle of formula (A⁶)

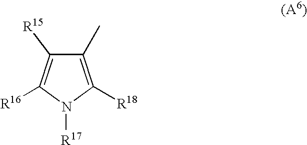

wherein:

R¹⁵ represents a hydrogen atom; a halogen atom; a cyano; C₁-C₅-alkyl or C₁-C₅-halogenoalkyl comprising up to 5 halogen atoms that can be the same or different;

R¹⁶ and R¹⁸ that can be the same or different represent a hydrogen atom; a halogen atom; C₁-C₅-alkoxycarbonyl; C₁-C₅-alkyl or C₁-C₅-halogenoalkyl comprising up to 5 halogen atoms that can be the same or different;

R¹⁷ represent a hydrogen atom or C₁-C₅-alkyl; C₁-C₅-halogenoalkyl comprising up to 5 halogen atoms that can be the same or different or a phenyl substituted by a halogen atom or by a C₁-C₅-alkyl;
a heterocycle of formula (A⁷)

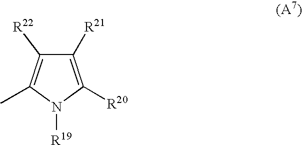

wherein:

R¹⁹ represents a hydrogen atom; C₁-C₅-alkyl or a phenyl substituted by a halogen atom or by a C₁-C₅-alkyl;

R²⁰ to R²² that can be the same or different represent a hydrogen atom; a halogen atom; C₁-C₅-alkyl or C₁-C₅-halogenoalkyl comprising up to 5 halogen atoms that can be the same or different;
a heterocycle of formula (A⁸)

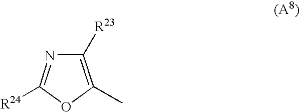

wherein:

R²³ represents a hydrogen atom; a halogen atom; C₁-C₅-alkyl; C₁-C₅-halogenoalkyl comprising up to 5 halogen atoms that can be the same or different;

R²⁴ represents a hydrogen atom or C₁-C₅-alkyl;
a heterocycle of formula (A⁹)

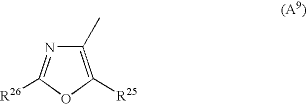

wherein:

R²⁵ represents a halogen atom; C₁-C₅-alkyl or C₁-C₅-halogenoalkyl comprising up to 5 halogen atoms that can be the same or different;

R²⁶ represents a hydrogen atom or C₁-C₅-alkyl;
a heterocycle of formula (A¹⁰)

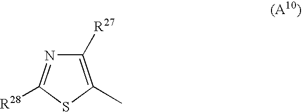

wherein:

R²⁷ represents a halogen atom; C₁-C₅-alkyl or C₁-C₅-halogenoalkyl comprising up to 5 halogen atoms that can be the same or different;

R²⁸ represents a hydrogen atom; a halogen atom; an amino; C₁-C₅-alkyl; C₁-C₅-halogenoalkyl comprising up to 5 halogen atoms that can be the same or different or a phenyl substituted by a halogen atom or by a C₁-C₅-alkyl;
a heterocycle of formula (A¹¹)

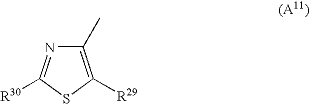

wherein:

R²⁹ represents a hydrogen atom; a halogen atom; C₁-C₅-alkyl or C₁-C₅-halogenoalkyl comprising up to 5 halogen atoms that can be the same or different;

R³⁰ represents a hydrogen atom; a halogen atom; C₁-C₅-alkyl; C₁-C₅-halogenoalkyl comprising up to 5 halogen atoms that can be the same or different; an amino; C₁-C₅-alkylamino; di-C₁ C₅-alkylamino;
a heterocycle of formula (A¹²)

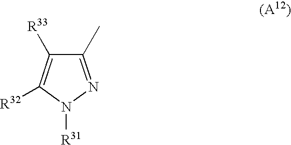

wherein:

R³¹ represents a hydrogen atom; a halogen atom; C₁-C₅-alkyl or a phenyl substituted by a halogen atom or by a C₁-C₅-alkyl;

R³² represents a hydrogen atom; a halogen atom; C₁-C₅-alkyl or C₁-C₅-halogenoalkyl comprising up to 5 halogen atoms that can be the same or different;

R³³ represents a hydrogen atom; a halogen atom; a nitro or C₁-C₅-halogenoalkyl comprising up to 5 halogen atoms that can be the same or different;
a heterocycle of formula (A¹³)

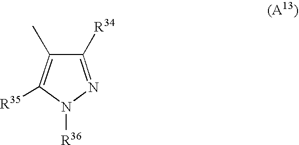

wherein:

R³⁴ represents a hydrogen atom; a halogen atom; C₁-C₅-alkyl; C₃-C₅-cycloalkyl; C₁-C₅-halogenoalkyl comprising up to 5 halogen atoms that can be the same or different; C₁-C₅-alkoxy; C₂-C₅-alkynyloxy; C₁-C₅-halogenoalkoxy comprising up to 5 halogen atoms that can be the same or different or a phenyl substituted by a halogen atom or by a C₁-C₅-alkyl;

R³⁵ represents a hydrogen atom; a halogen atom; C₁-C₅-alkyl; a cyano; C₁-C₅-alkoxy; C₁-C₅-alkylsulfanyl; C₁-C₅-halogenoalkyl comprising up to 5 halogen atoms that can be the same or different; C₁-C₅-halogenoalkoxy comprising up to 5 halogen atoms that can be the same or different; an amino; C₁-C₅-alkylamino or di(C₁-C₅-alkyl)-amino;

R³⁶ represents a hydrogen atom; C₁-C₅-alkyl or a phenyl substituted by a halogen atom or by a C₁-C₅-alkyl;
a heterocycle of formula (A¹⁴)

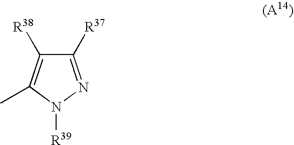

wherein:

R³⁷ and R³⁸ that can be the same or different represent a hydrogen atom; a halogen atom; C₁-C₅-alkyl; C₁-C₅-halogenoalkyl comprising up to 5 halogen atoms that can be the same or different; C₁-C₅-alkoxy or a C₁-C₅-alkylsulfanyl;

R³⁹ represents a hydrogen atom; C₁-C₅-alkyl or a phenyl substituted by a halogen atom or by a C₁-C₅-alkyl;
a heterocycle of formula (A¹⁵)

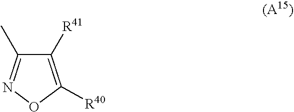

wherein R⁴⁰ and R⁴¹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A¹⁶)

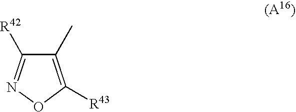

wherein R⁴² and R⁴³ that can be the same or different represent a hydrogen atom; C₁-C₅-alkyl; C₁-C₅-halogenoalkyl comprising up to 5 halogen atoms that can be the same or different; an amino or a phenyl substituted by a halogen atom or by a C₁-C₅-alkyl;
a heterocycle of formula (A¹⁷)

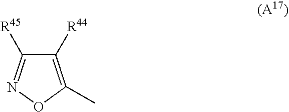

wherein R⁴⁴ and R⁴⁵ that can be the same or different represent a hydrogen atom; a halogen atom; C₁-C₅-alkyl or C₁-C₅-halogenoalkyl comprising up to 5 halogen atoms that can be the same or different;
a heterocycle of formula (A¹⁸)

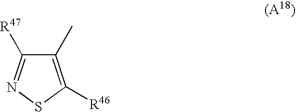

wherein:

R⁴⁶ represents a hydrogen atom; C₁-C₅-alkyl; C₁-C₅-halogenoalkyl comprising up to 5 halogen atoms that can be the same or different or C₁-C₅-alkylsulfanyl;

R⁴⁷ represents a hydrogen atom; a halogen atom or C₁-C₅-alkyl;
a heterocycle of formula (A¹⁹)

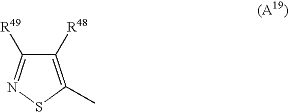

wherein R⁴⁸ and R⁴⁹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²⁰)

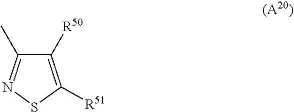

wherein R⁵⁰ and R⁵¹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²¹)

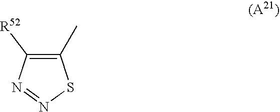

wherein R⁵² represents a halogen atom; C₁-C₅-alkyl or C₁-C₅-halogenoalkyl comprising up to 5 halogen atoms that can be the same or different;
a heterocycle of formula (A²²)

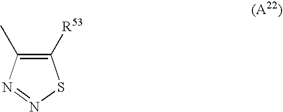

wherein R⁵³ represents a halogen atom; C₁-C₅-alkyl or C₁-C₅-halogenoalkyl comprising up to 5 halogen atoms that can be the same or different;
a heterocycle of formula (A²³)

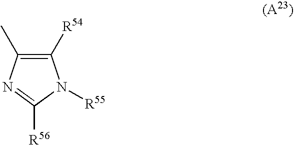

wherein:

R⁵⁴ and R⁵⁶ that can be the same or different represent a hydrogen atom; a halogen atom; C₁-C₅-alkyl or C₁-C₅-halogenoalkyl comprising up to 5 halogen atoms that can be the same or different;

R⁵⁵ represents a hydrogen atom or C₁-C₅-alkyl;
a heterocycle of formula (A²⁴)

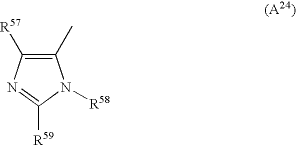

wherein:

R⁵⁷ and R⁵⁹ that can be the same or different represent a hydrogen atom; a halogen atom; C₁-C₅-alkyl or C₁-C₅-halogenoalkyl comprising up to 5 halogen atoms that can be the same or different;

R⁵⁸ represents a hydrogen atom or C₁-C₅-alkyl;
a heterocycle of formula (A²⁵)

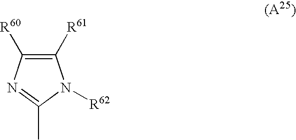

wherein:

R⁶⁰ and R⁶¹ that can be the same or different represent a hydrogen atom; a halogen atom; C₁-C₅-alkyl or C₁-C₅-halogenoalkyl comprising up to 5 halogen atoms that can be the same or different;

R⁶² represents a hydrogen atom or C₁-C₅-alkyl;
a heterocycle of formula (A²⁶)

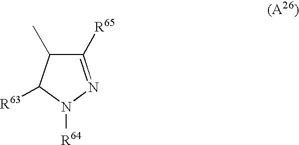

wherein:

R⁶⁵ represents a hydrogen atom; a halogen atom; C₁-C₅-alkyl; C₃-C₅-cycloalkyl; C₁-C₅-halogenoalkyl comprising up to 5 halogen atoms that can be the same or different; C₁-C₅-alkoxy; C₂-C₅-alkynyloxy or C₁-C₅-halogenoalkoxy comprising up to 5 halogen atoms that can be the same or different;

R⁶³ represents a hydrogen atom; a halogen atom; C₁-C₅-alkyl; a cyano; C₁-C₅-alkoxy; C₁-C₅-alkylsulphanyl; C₁-C₅-halogenoalkyl comprising up to 5 halogen atoms that can be the same or different; C₁-C₅-halogenoalkoxy comprising up to 5 halogen atoms that can be the same or different; amino; C₁-C₅alkylamino or di(C₁-C₅-alkyl)amino;

R⁶⁴ represents a hydrogen atom or C₁-C₅-alkyl.

More preferred compounds of formula (I) according to the invention are those wherein A represents a 5-membered heterocycle that is substituted in position ortho. Such compounds can also be defined with A representing an α-substituted 5-membered heterocycle.

Still more preferred compounds of formula (I) according to the invention are those wherein A represents A¹³.

Still other preferred compounds of formula (I) according to the invention are those wherein T represents sulphur.

Other preferred compounds of formula (I) according to the invention are those wherein Z¹ represents a non-substituted C₃-C₇ cycloalkyl, more preferably cyclopropyl.

Other preferred compounds of formula (I) according to the invention are those wherein Z² and Z³, that can be the same or different, represent a hydrogen atom or C₁-C₅-alkyl.

Still other preferred compounds of formula (I) according to the invention are those wherein Y represents trifluoromethyl.

Still other preferred compounds of formula (I) according to the invention are those wherein X, that can be the same or different, represents a halogen atom; C₁-C₈-alkyl; C₁-C₆-halogenoalkyl comprising up to 5 halogen atoms that can be the same or different; C₁-C₈-alkoxy; C₁-C₆-halogenoalkoxy comprising up to 5 halogen atoms that can be the same or different.

Still other preferred compounds of formula (I) according to the invention are those wherein n represents 1.

The above mentioned preferences with regard to the substituents of the compounds according to the invention can be combined in various manners. These combinations of preferred features thus provide sub-classes of compounds according to the invention. Examples of such sub-classes of preferred compounds according to the invention can be combined:
preferred features of A with preferred features of T, Z¹, Z², Z³, Y, X and n;
preferred features of T with preferred features of A, Z¹, Z², Z³, Y, X and n;
preferred features of Z¹ with preferred features of A, T, Z², Z³, Y, X and n;
preferred features of Z² with preferred features of A, T, Z¹, Z³, Y, X and n;
preferred features of Z³ with preferred features of A, T, Z¹, Z², Y, X and n;
preferred features of Y with preferred features of A, T, Y, Z¹, Z², Z³, Y, X and n;
preferred features of X with preferred features of A, T, Y, Z¹, Z², Z³, Y and n;
preferred features of n with preferred features of A, Y, Z¹, Z², Z³, Y and X.

In these combinations of preferred features of the substituents of the compounds according to the invention, the said preferred features can also be selected among the more preferred features of each of A, T, Z¹, Z², Z³, Y, X and n so as to form most preferred subclasses of compounds according to the invention.

The present invention also relates to a process for the preparation of compounds of formula (I). Thus according to a further aspect of the present invention there is provided a process P1 for the preparation of compound of formula (I) wherein T represents S, as illustrated by the following reaction scheme:

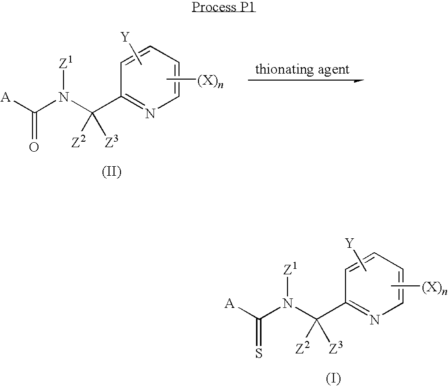

wherein A, Z¹ to Z³, Y, X and n are as herein-defined.

Process P1 can be performed in the presence of a thionating agent.

Amide derivatives of formula (II) are known or can be prepared by known processes, for example as described in European patent application EP-06/356008.

Suitable thionating agents for carrying out process P1 according to the invention can be sulphur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in J. Chem. Soc. Perkin 1, (2001), 358,

In the presence or in the absence, of a catalytic or stoechiometric or more, quantity of a base such as an inorganic or an organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethyl-aminopyridine or N-methylpiperidine.

Suitable solvents for carrying out process P1 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane or 1,2-diethoxyethane; nitriles, such as acetonitrile, propionitrile, n-or i-butyronitrile or benzonitrile; sulphurous solvents, such as sulpholane or carbon disulfide.

When carrying out process P1 according to the invention, the reaction temperatures can be varied within a relatively wide range. In general, these processes are carried out at temperatures from 0° C. to 160° C., preferably from 10° C. to 120° C. A way to control the temperature for the processes according to the invention is to use micro-wave technology.

Process P1 according to the invention is generally carried out under atmospheric pressure. It is also possible to operate under elevated or reduced pressure.

When carrying out process P1 according to the invention, 1 mole or an excess of the sulphur equivalent of the thionating agent and from 1 to 3 moles of the base can be employed per mole of the amide derivative of formula (II).

It is also possible to employ the reaction components in other ratios. Work-up is carried out by known methods.

In general, the reaction mixture is concentrated under reduced pressure. The residue that remains can be freed by known methods, such as chromatography or recrystallization, from any impurities that can still be present.

According to a further aspect according to the invention, there is provided a process P2 for the preparation of a compound of formula (I), wherein T is selected in the list consisting of N—R^{a}, N—OR^{a}, N—NR^{a}R^{b} or N—CN, as illustrated by the following reaction scheme:

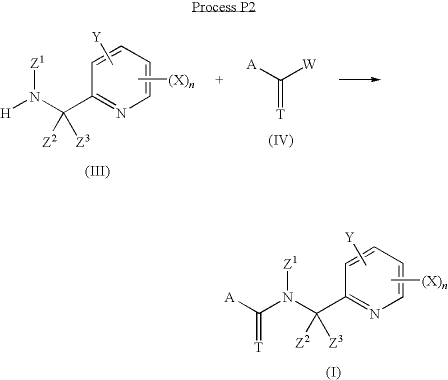

wherein
A, Z¹ to Z³, R^{a}, R^{b}, Y, X and n are as herein-defined;
W represents a chlorine atom or a methylsulfanyl group.

Process P2 can be performed in the presence of an acid binder or in the presence of a solvent.

Amine derivatives of formula (III) are known or can be prepared by known processes or can be prepared by known processes, for example as described in WO-2001/11966 pages 20, 21 and 23.

N-substituted carboximidoyl chloride of formula (IV) are known or can be prepared by known processes, for example as described in Houben-Weyl, “Methoden der organischen Chemie” (1985), E5/1, 628-633 and Patai, “The chemistry of amidines and imidates” (1975), 296-301.

N-substituted or N,N-disubstituted hydrazonoyl chloride of formula (IV) are known or can be prepared by known processes, for example as described in Tetrahedron, 1991, 47, p 447 and Journal of Heterocyclic Chemistry, 1983, 20, p 225.

N-cyano carboximidoyl chloride of formula (IV) are known or can be prepared by known processes, for example as described in Tetrahedron Letters, 1968, p 5523 and Bioorganic and Medicinal Chemistry, 2006, p 4723.

Suitable acid binders for carrying out process P2 according to the invention can be inorganic and organic bases that are customary for such reactions. Preference is given to using alkaline earth metal or alkali metal hydroxides, such as sodium hydroxide, calcium hydroxide, potassium hydroxide or other ammonium hydroxide derivatives; alkali metal carbonates, such as sodium carbonate, potassium carbonate, potassium bicarbonate, sodium bicarbonate; alkali metal or alkaline earth metal acetates, such as sodium acetate, potassium acetate, calcium acetate; alkaline earth metal or alkali metal hydrides, such as sodium hydride or potassium hydride; alkaline earth metal or alkali metal alcoolates, such as sodium methylate, sodium ethylate, sodium propylate or potassium t-butylate; and also tertiary amines, such as trimethylamine, triethylamine, tributylamine, N,N-dimethylaniline, pyridine, N-methylpiperidine, N,N-dimethyl-aminopyridine, diazabicyclooctane (DABCO), diazabicyclononene (DBN) or diaza-bicycloundecene (DBU); or a polymer-supported acid scavenger (for example as detailed in http://www.iris-biotech.de/downloads/scavengers.pdf).

It is also possible to work in the absence of any additional acid binder.

Suitable solvents for carrying out process P2 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n-or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide, or sulphones, such as sulpholane.

When carrying out process P2 according to the invention, the reaction temperatures can be varied within a relatively wide range. In general, these processes are carried out at temperatures from 0° C. to 160° C., preferably from 10° C. to 120° C. A way to control the temperature for the processes according to the invention is to use micro-wave technology.

Process P2 according to the invention is generally carried out under atmospheric pressure. It is also possible to operate under elevated or reduced pressure.

When carrying out process P2 according to the invention, the amine derivative of formula (III) can be employed as its salt, such as chlorhydate or any other convenient salt.

When carrying out process P2 according to the invention, 1 mole or an excess of the amine derivative of formula (III) and from 1 to 3 moles of the acid binder can be employed per mole of the N-substituted carboximidoyl chloride of formula (IV).

It is also possible to employ the reaction components in other ratios. Work-up is carried out by known methods.

In general, the reaction mixture is concentrated under reduced pressure. The residue that remains can be freed by known methods, such as chromatography or recrystallization, from any impurities that can still be present.

Compounds of formula (I) according to the invention can be prepared according to the herein described processes. It will nevertheless be understood that, on the basis of his general knowledge and of available publications, the skilled worker will be able to adapt these processes according to the specifics of each of the compounds that it is desired to be synthesised.

In a further aspect, the present invention also relates to a fungicide composition comprising an effective and non-phytotoxic amount of an active compound of formula (I).

The expression “effective and non-phytotoxic amount” means an amount of composition according to the invention that is sufficient to control or destroy the fungi present or liable to appear on the crops, and that does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials, that are within the capabilities of a person skilled in the art.

Thus, according to the invention, there is provided a fungicide composition comprising, as an active ingredient, an effective amount of a compound of formula (I) as herein defined and an agriculturally acceptable support, carrier or filler.

According to the invention, the term “support” denotes a natural or synthetic, organic or inorganic compound with that the active compound of formula (I) is combined or associated to make it easier to apply, notably to the parts of the plant. This support is thus generally inert and should be agriculturally acceptable. The support can be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports can also be used.

The composition according to the invention can also comprise additional components. In particular, the composition can further comprise a surfactant. The surfactant can be an emulsifier, a dispersing agent or a wetting agent of ionic or non-ionic type or a mixture of such surfactants. Mention can be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active compound and/or the inert support are water-insoluble and when the vector agent for the application is water. Preferably, surfactant content can be comprised from 5% to 40% by weight of the composition.

Optionally, additional components can also be included, e.g. protective colloids, adhesives, thickeners, thixotropic agents, penetration agents, stabilisers, sequestering agents. More generally, the active compounds can be combined with any solid or liquid additive, that complies with the usual formulation techniques.

In general, the composition according to the invention can contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions that are ready to be applied to the plant or seed to be treated by means of a suitable device, such as a spraying or dusting device, but also concentrated commercial compositions that must be diluted before application to the crop.

The compounds according to the invention can also be mixed with one or more insecticide, fungicide, bactericide, attractant, acaricide or pheromone active substance or other compounds with biological activity. The mixtures thus obtained have normally a broadened spectrum of activity. The mixtures with other fungicide compounds are particularly advantageous.

Examples of suitable fungicide mixing partners can be selected in the following lists:
(1) Inhibitors of the nucleic acid synthesis, for example benalaxyl, benalaxyl-M, bupirimate, clozylacon, dimethirimol, ethirimol, furalaxyl, hymexazol, metalaxyl, metalaxyl-M, ofurace, oxadixyl and oxolinic acid.
(2) Inhibitors of the mitosis and cell division, for example benomyl, carbendazim, chlorfenazole, diethofencarb, ethaboxam, fuberidazole, pencycuron, thiabendazole, thiophanate, thiophanate-methyl and zoxamide.
(3) Inhibitors of the respiration, for example diflumetorim as CI-respiration inhibitor; bixafen, boscalid, carboxin, fenfuram, flutolanil, fluopyram, furametpyr, furmecyclox, isopyrazam (9R-component), isopyrazam (9S-component), mepronil, oxycarboxin, penthiopyrad, thifluzamide as CII-respiration inhibitor; amisulbrom, azoxystrobin, cyazofamid, dimoxystrobin, enestroburin, famoxadone, fenamidone, fluoxastrobin, kresoxim-methyl, metominostrobin, orysastrobin, picoxystrobin, pyraclostrobin, pyribencarb, trifloxystrobin as CIII-respiration inhibitor.
(4) Compounds capable to act as an uncoupler, like for example binapacryl, dinocap, fluazinam and meptyldinocap.
(5) Inhibitors of the ATP production, for example fentin acetate, fentin chloride, fentin hydroxide, and silthiofam.
(6) Inhibitors of the amino acid and/or protein biosynthesis, for example andoprim, blasticidin-S, cyprodinil, kasugamycin, kasugamycin hydrochloride hydrate, mepanipyrim and pyrimethanil.
(7) Inhibitors of the signal transduction, for example fenpiclonil, fludioxonil and quinoxyfen.
(8) Inhibitors of the lipid and membrane synthesis, for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl and vinclozolin.
(9) Inhibitors of the ergosterol biosynthesis, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, viniconazole and voriconazole.
(10) Inhibitors of the cell wall synthesis, for example benthiavalicarb, dimethomorph, flumorph, iprovalicarb, mandipropamid, polyoxins, polyoxorim, prothiocarb, validamycin A, and valiphenal.
(11) Inhibitors of the melanine biosynthesis, for example carpropamid, diclocymet, fenoxanil, phthalide, pyroquilon and tricyclazole.
(12) Compounds capable to induce a host defence, like for example acibenzolar-S-methyl, probenazole, and tiadinil.
(13) Compounds capable to have a multisite action, like for example bordeaux mixture, captafol, captan, chlorothalonil, copper naphthenate, copper oxide, copper oxychloride, copper preparations such as copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulphur and sulphur preparations including calcium polysulphide, thiram, tolylfluanid, zineb and ziram.
(14) Further compounds like for example 2,3-dibutyl-6-chlorothieno[2,3-d]pyrimidin-4(3H)-one, ethyl (2Z)-3-amino-2-cyano-3-phenylprop-2-enoate, N-[2-(1,3-dimethylbutyl)phenyl]-5-fluoro-1,3-dimethyl-1H-pyrazole-4-carboxamide, N-{2-[1,1′-bi(cyclopropyl)-2-yl]phenyl}-3-(difluoromethyl)-1-methyl-1H-pyrazole-4-carboxamide, (2E)-2-(2-{[6-(3-chloro-2-methylphenoxy)-5-fluoropyrimidin-4-yl]oxy}phenyl)-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, N-(3-ethyl-3,5,5-trimethylcyclohexyl)-3-(formylamino)-2-hydroxybenzamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N-ethyl-N-methyl-N′-{2-methyl-5-(trifluoromethyl)-4-[3-(trimethylsilyl)propoxy]phenyl}imidoformamide, N′-{5-(difluoromethyl)-2-methyl-4-[3-(trimethylsilyl)propoxy]phenyl}-N-ethyl-N-methylimidoformamide, O-{1-[(4-methoxyphenoxy)methyl]-2,2-dimethylpropyl} 1H-imidazole-1-carbothioate, N-[2-(4-{[3-(4-chlorophenyl)prop-2-yn-1-yl]oxy}-3-methoxyphenyl)ethyl]-N2-(methylsulfonyl)valinamide, 5-chloro-7-(4-methylpiperid in-1-yl)-6-(2,4,6-trifluorophenyl)[1,2,4]triazolo[1,5-a]pyrimidine, 5-amino-1,3,4-thiadiazole-2-thiol, propamocarb-fosetyl, 1-[(4-methoxyphenoxy)methyl]-2,2-dimethylpropyl 1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulfonyl)pyridine, 2-butoxy-6-iodo-3-propyl-4H-chromen-4-one, 2-phenylphenol and salts, 3-(difluoromethyl)-1-methyl-N-[2-(1,1,2,2-tetrafluoroethoxy)phenyl]-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, quinolin-8-ol, quinolin-8-ol sulfate (2:1) (salt), benthiazole, bethoxazin, capsimycin, carvone, chinomethionat, chloroneb, cufraneb, cyflufenamid, cymoxanil, cyprosulfamide, dazomet, debacarb, dichlorophen, diclomezine, dicloran, difenzoquat, difenzoquat methylsulphate, diphenylamine, ecomate, ferimzone, flu metover, fluopicolide, fluoroimide, flusulfamide, fosetyl-aluminium, fosetyl-calcium, fosetyl-sodium, hexachlorobenzene, irumamycin, isotianil, methasulfocarb, methyl (2E)-2-{2-[({cyclopropyl[(4-methoxyphenyl)imino]methyl}thio)methyl]phenyl}-3-methoxyacrylate, methyl isothiocyanate, metrafenone, (5-bromo-2-methoxy-4-methylpyridin-3-yl)(2,3,4-trimethoxy-6-methylphenyl)methanone, mildiomycin, tolnifanide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(Z)-[(cyclopropylmethoxy)imino][6-(difluoromethoxy)-2,3-difluorophenyl]methyl}-2-phenylacetamide, N-{(E)-[(cyclopropylmethoxy)imino][6-(difluoromethoxy)-2,3-difluorophenyl]methyl}-2-phenylacetamide, natamycin, nickel dimethyldithiocarbamate, nitrothal-isopropyl, octhilinone, oxamocarb, oxyfenthiin, pentachlorophenol and salts, phenazine-1-carboxylic acid, phenothrin, phosphorous acid and its salts, propamocarb fosetylate, propanosine-sodium, proquinazid, pyrroInitrine, quintozene, S-prop-2-en-1-yl 5-amino-2-(1-methylethyl)-4-(2-methylphenyl)-3-oxo-2,3-dihydro-1H-pyrazole-1-carbothioate, tecloftalam, tecnazene, triazoxide, trichlamide, 5-chloro-N′-phenyl-N′-prop-2-yn-1-ylthiophene-2-sulfonohydrazide and zarilamid.

The composition according to the invention comprising a mixture of a compound of formula (I) with a bactericide compound can also be particularly advantageous. Examples of suitable bactericide mixing partners can be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The compounds of formula (I) and the fungicide composition according to the invention can be used to curatively or preventively control the phytopathogenic fungi of plants or crops. Thus, according to a further aspect of the invention, there is provided a method for curatively or preventively controlling the phytopathogenic fungi of plants or crops characterised in that a compound of formula (I) or a fungicide composition according to the invention is applied to the seed, the plant or to the fruit of the plant or to the soil wherein the plant is growing or wherein it is desired to grow.

The method of treatment according to the invention can also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the overground parts of the plant such as trunks, stems or stalks, leaves, flowers and fruit of the concerned plant.

Among the plants that may becan be protected by the method according to the invention, mention may becan be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantins), Rubiaceae sp., Theaceae sp., Sterculiceae sp., Rutaceae sp. (for instance lemons oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rye, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots), Elaeis sp. (for instance oil palm); horticultural and forest crops; as well as genetically modified homologues of these crops.

Among the diseases of plants or crops that can be controlled by the method according to the invention, mention can be made of:

Powdery Mildew Diseases such as
Blumeria diseases caused for example by Blumeria graminis;
Podosphaera diseases caused for example by Podosphaera leucotricha;
Sphaerotheca diseases caused for example by Sphaerotheca fuliginea;
Uncinula diseases caused for example by Uncinula necator ;

Rust Diseases such as
Gymnosporangium diseases caused for example by Gymnosporangium sabinae;
Hemileia diseases caused for example by Hemileia vastatrix ;
Phakopsora diseases caused for example by Phakopsora pachyrhizi and Phakopsora meibomiae;
Puccinia diseases caused for example by Puccinia recondita, Puccinia graminis or Puccinia striiformis ;
Uromyces diseases caused for example by Uromyces appendiculatus ;

Oomycete Diseases such as
Albugo diseases caused for example by Albugo candida;
Bremia diseases caused for example by Bremia lactucae;
Peronospora diseases caused for example by Peronospora pisi and Peronospora brassicae;
Phytophthora diseases caused for example by Phytophthora infestans;
Plasmopara diseases caused for example by Plasmopara viticola;
Pseudoperonospora diseases caused for example by Pseudoperonospora humuli and Pseudoperonospora cubensis;
Pythium diseases caused for example by Pythium ultimum ;

Leaf spot, Leaf blotch and Leaf Blight Diseases such as
Alternaria diseases caused for example by Alternaria solani;
Cercospora diseases caused for example by Cercospora beticola;
Cladiosporium diseases caused for example by Cladiosporium cucumerinum;
Cochliobolus diseases caused for example by Cochliobolus sativus (Conidiaform: Drechslera, Syn: Helminthosporium) or Cochliobolus miyabeanus;
Colletotrichum diseases caused for example by Colletotrichum lindemuthianum;
Cycloconium diseases caused for example by Cycloconium oleaginum;
Diaporthe diseases caused for example by Diaporthe citri;
Elsinoe diseases caused for example by Elsinoe fawcettii;
Gloeosporium diseases caused for example by Gloeosporium laeticolor;
Glomerella diseases caused for example by Glomerella cingulata;
Guignardia diseases caused for example by Guignardia bidwelfii;
Leptosphaeria diseases caused for example by Leptosphaeria maculans and Leptosphaeria nodorum;
Magnaporthe diseases caused for example by Magnaporthe grisea;
Mycosphaerella diseases caused for example by Mycosphaerella graminicola,
Mycosphaerella arachidicola and Mycosphaerella fijiensis;
Phaeosphaeria diseases caused for example by Phaeosphaeria nodorum;
Pyrenophora diseases caused for example by Pyrenophora teres or Pyrenophora tritici repentis;
Ramularia—diseases caused for example by Ramularia collo-cygni or Ramularia areola;
Rhynchosporium diseases caused for example by Rhynchosporium secalis;
Septoria diseases caused for example by Septoria apii and Septoria lycopersici;
Typhula diseases caused for example by Thyphula incamata;
Venturia diseases caused for example by Venturia inaequalis ;

Root-, Sheath and Stem Diseases such as
Corticium diseases caused for example by Corticium graminearum;
Fusarium diseases caused for example by Fusarium oxysporum;
Gaeumannomyces diseases caused for example by Gaeumannomyces graminis;
Rhizoctonia diseases caused for example by Rhizoctonia solani;
Sarocladium diseases caused for example by Sarocladium oryzae;
Sclerotium diseases caused for example by Sclerotium oryzae;
Tapesia diseases caused for example by Tapesia acuformis;
Thielaviopsis diseases caused for example by Thielaviopsis basicola;

Ear and Panicle Diseases including Maize cob such as
Alternaria diseases caused for example by Alternaria spp.;
Aspergillus diseases caused for example by Aspergillus flavus;
Cladosporium diseases caused for example by Cladiosporium cladosporioides;
Claviceps diseases caused for example by Claviceps purpurea;
Fusarium diseases caused for example by Fusarium culmorum;
Gibberella diseases caused for example by Gibberella zeae;
Monographella diseases caused for example by Monographella nivalis ;

Smut- and Bunt Diseases such as
Sphacelotheca diseases caused for example by Sphacelotheca reiliana;
Tilletia diseases caused for example by Tilletia caries;
Urocystis diseases caused for example by Urocystis occulta;
Ustilago diseases caused for example by Ustilago nuda ;

Fruit Rot and Mould Diseases such as
Aspergillus diseases caused for example by Aspergillus flavus;
Botrytis diseases caused for example by Botrytis cinerea;
Penicillium diseases caused for example by Penicillium expansum and Penicillium purpurogenum;
Rhizopus diseases caused by example by Rhizopus stolonifer
Sclerotinia diseases caused for example by Sclerotinia sclerotiorum;
Verticillium diseases caused for example by Verticillium alboatrum;

Seed- and Soilborne Decay, Mould, Wilt, Rot and Damping-off diseases
Alternaria diseases caused for example by Alternaria brassicicola;
Aphanomyces diseases caused for example by Aphanomyces uteiches;
Ascochyta diseases caused for example by Ascochyta lentis;
Aspergillus diseases caused for example by Aspergillus flavus;
Cladosporium diseases caused for example by Cladosporium herbarum;
Cochliobolus diseases caused for example by Cochliobolus sativus; (Conidiaform: Drechslera, Bipolaris Syn: Helminthosporium);
Colletotrichum diseases caused for example by Colletotrichum coccodes;
Fusarium diseases caused for example by Fusarium culmorum;
Gibberella diseases caused for example by Gibberella zeae ;
Macrophomina diseases caused for example by Macrophomina phaseolina;
Microdochium diseases caused for example by Microdochium nivale;
Monographella diseases caused for example by Monographella nivalis;
Penicillium diseases caused for example by Penicillium expansum;
Phoma diseases caused for example by Phoma lingam;
Phomopsis diseases caused for example by Phomopsis sojae;
Phytophthora diseases caused for example by Phytophthora cactorum;
Pyrenophora diseases caused for example by Pyrenophora graminea;
Pyricularia diseases caused for example by Pyricularia oryzae;
Pythium diseases caused for example by Pythium ultimum;
Rhizoctonia diseases caused for example by Rhizoctonia solani;
Rhizopus diseases caused for example by Rhizopus oryzae;
Sclerotium diseases caused for example by Sclerotium rolfsii;
Septoria diseases caused for example by Septoria nodorum;
Typhula diseases caused for example by Typhula incamata;
Verticillium diseases caused for example by Verticillium dahliae;

Canker, Broom and Dieback Diseases such as
Nectria diseases caused for example by Nectria galligena;

Blight Diseases such as
Monilinia diseases caused for example by Monilinia laxa;

Leaf Blister or Leaf Curl Diseases including deformation of blooms and fruit such as
Exobasidium diseases caused for example by Exobasidium vexans.
Taphrina diseases caused for example by Taphrina deformans;

Decline Diseases of Wooden Plants such as
Esca disease caused for example by Phaeomoniella clamydospora, Phaeoacremonium aleophilum and Fomitiporia mediterranea;
Ganoderma diseases caused by example by Ganoderma boninense;

Diseases of Flowers and Seeds such as
Botrytis diseases caused for example by Botrytis cinerea ;

Diseases of Tubers such as
Rhizoctonia diseases caused for example by Rhizoctonia solani;
Helminthosporium diseases caused for example by Helminthosporium solani;

Club root diseases such as
Plasmodiophora diseases, caused for example by Plamodiophora brassicae;

Diseases caused by Bacterial Organisms such as
Xanthomanas species for example Xanthomonas campestris pv. oryzae;
Pseudomonas species for example Pseudomonas syringae pv. lachrymans;
Erwinia species for example Erwinia amylovora.

The fungicide composition according to the invention can also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention, or a composition according to the invention; this includes for example direct application, spraying, dipping, injection or any other suitable means.

The dose of active compound usually applied in the method of treatment according to the invention is generally and advantageously from 10 to 800 g/ha, preferably from 50 to 300 g/ha for applications in foliar treatment. The dose of active substance applied is generally and advantageously from 2 to 200 g per 100 kg of seed, preferably from 3 to 150 g per 100 kg of seed in the case of seed treatment.

It is clearly understood that the doses indicated herein are given as illustrative examples of the method according to the invention. A person skilled in the art will know how to adapt the application doses, notably according to the nature of the plant or crop to be treated.

The fungicide composition according to the invention can also be used in the treatment of genetically modified organisms with the compounds according to the invention or the agrochemical compositions according to the invention. Genetically modified plants are plants into genome of that a heterologous gene encoding a protein of interest has been stably integrated. The expression “heterologous gene encoding a protein of interest” essentially means genes that give the transformed plant new agronomic properties, or genes for improving the agronomic quality of the modified plant.

The compounds or mixtures according to the invention can also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

The various aspects of the invention will now be illustrated with reference to the following table of compound examples and the following preparation example.

The table 1 illustrates in a non-limiting manner examples of compounds according to the invention. In table 1, M+H (or M−H) means the molecular ion peak, plus or minus 1 a.m.u. (atomic mass unit) respectively, as observed in mass spectroscopy and M (Apcl+) means the molecular ion peak as it was found via positive atmospheric pressure chemical ionisation in mass spectroscopy.

TABLE 1

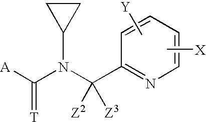

| Example | A | X | Y | Z² | Z³ | T | logP | M + 1 |
|---|---|---|---|---|---|---|---|---|
| 1 | 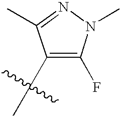 | 3-Cl | 5-CF₃ | H | H | S | 3.65 | 407 |
| 2 | 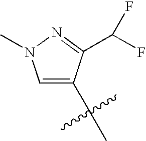 | 3-Cl | 5-CF₃ | H | H | S | 3.69 | 425 |
| 3 | 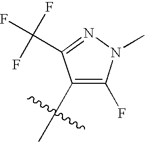 | 3-Cl | 5-CF₃ | H | H | S | 4.41 | 461 |

The following example illustrate in a non-limiting manner the preparation of the compounds of formula (I) according to the invention.

### General Preparation Example: Thionation of Amide of Formula (I) on Chemspeed Apparatus

In a 13 ml Chemspeed vial is weighted 0.27 mmole of phosphorous pentasulfide (P₂S₅). 3 ml of a 0.18 molar solution of the amide (I) (0.54 mmole) in dioxane is added and the mixture is heated at reflux for two hours. The temperature is then cooled to 80° C. and 2.5 ml of water are added. The mixture is heated at 80° C. for one more hour. 2 ml of water are then added and the reaction mixture is extracted twice by 4 ml of dichloromethane. The organic phase is deposited on a basic alumina cardridge (2 g) and eluted twice by 8 ml of dichloromethane. The solvents are removed and the crude thioamide derivative is analyzed by LCMS and NMR. Insufficiently pure compounds are further purified by preparative LCMS.

### EXAMPLE A

### In Vivo Test on Pyrenophora teres (Barley Net Blotch)

The active ingredients tested are prepared by homogenization in a mixture of acetone/tween/DMSO, then diluted with water to obtain the desired active material concentration.

Barley plants (Express variety), sown on a 50/50 peat soil-pozzolana substrate in starter cups and grown at 12° C., are treated at the 1-leaf stage (10 cm tall) by spraying with the active ingredient prepared as described above. Plants, used as controls, are treated with the mixture of acetone/tween/DMSO/water not containing the active material.

After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants. Under these conditions, good (at least 70%) or total protection is observed at a dose of 500 ppm with the following compounds: 1, 2 and 3.

### EXAMPLE B

### In Vivo Test on Sphaerotheca Fuliginea (Powdery Mildew)

The active ingredients tested are prepared by homogenization in a mixture of acetone/tween/water. This suspension is then diluted with water to obtain the desired active material concentration.

Gherkin plants (Vert petit de Paris variety) in starter cups, sown on a 50/50 peat soil-pozzolana substrate and grown at 20° C./23° C., are treated at the cotyledon Z10 stage by spraying with the aqueous suspension described above. Plants, used as controls, are treated with an aqueous solution not containing the active material.

After 24 hours, the plants are contaminated by spraying them with an aqueous suspension of Sphaerotheca fuliginea spores (100 000 spores per ml). The spores are collected from a contaminated plants. The contaminated gherkin plants are incubated at about 20° C./25° C. and at 60/70% relative humidity.

Grading (% of efficacy) is carried out 12 days after the contamination, in comparison with the control plants.

Under these conditions, good (at least 70%) or total protection is observed at a dose of 500 ppm with the following compounds: 1, 2 and 3.

### EXAMPLE C

### In Vivo Test on Puccinia recondita (Brown Rust)

The active ingredients tested are prepared by homogenization in a mixture of acetone/tween/DMSO, then diluted with water to obtain the desired active material. Wheat plants (Scipion variety) sown on 50/50 peat soil-pozzolana substrate in starter cups and grown at 12° C., are treated at the 1-leaf stage (10 cm tall) by spraying with the aqueous suspension described above.

Plants, used as controls, are treated with an aqueous solution not containing the active material.

After 24 hours, the plants are contaminated by spraying the leaves with an aqueous suspension of Puccinia recondita spores (100,000 spores per ml). The spores are collected from a 10-day-old contaminated wheat and are suspended in water containing 2.5 ml/l of tween 80 10%. The contaminated wheat plants are incubated for 24 hours at 20° C. and at 100% relative humidity, and then for 10 days at 20° C. and at 70% relative humidity. Grading is carried out 10 days after the contamination, in comparison with the control plants. Under these conditions, good (at least 70%) or total protection is observed at a dose of 500 ppm with the following compounds: 3.

1. A compound of formula (I) 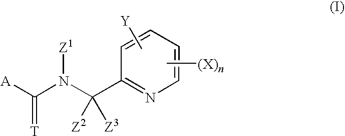
wherein
A represents a carbo-linked, substituted or non substituted, unsaturated or partially saturated, 5-membered heterocyclyl group;
T represents S, N—R^{a}, N—OR^{a}, N—NR^{a}R^{b} or N—CN;
Z¹ represents a substituted or non substituted C₃-C₇ cycloalkyl;
Z² and Z³, that can be the same or different, represent a hydrogen atom; C₁-C₅-alkyl; C₂-C₅-alkenyl; C₂-C₅-alkynyl; cyano; nitro; a halogen atom; C₁-C₅-alkoxy; C₂ C₅-alkenyloxy; C₂-C₅-alkynyloxy; C₃-C₇-cycloalkyl; C₁-C₅-alkylsulphenyl; an amino; C₁-C₅-alkylamino; di-C₁ C₅-alkylamino; C₁-C₅-alkoxycarbonyl; C₁-C₅-alkylcarbamoyl; di-C₁-C₅-alkylcarbamoyl; N—C₁-C₅-alkyl-C₁-C₅-alkoxycarbamoyl; or
Z² and Z³ together with the carbon atom to that they are linked can form a 3-, 4-, 5-or 6-membered carbo-or heterocyclic ring, that can be substituted;
Y represents a C₁-C₅-halogenoalkyl comprising up to 9 halogen atoms that can be the same or different;
X, that can be the same or different, represents a halogen atom; nitro; cyano; hydroxyl; a carboxyl group; C₁-C₈-alkyl; C₁-C₆-halogenoalkyl comprising up to 9 halogen atoms that can be the same or different; C₁-C₈-alkylamino; di-C₁-C₈-alkylamino; C₁-C₈-alkoxy; C₁-C₆-halogenoalkoxy comprising up to 9 halogen atoms that can be the same or different; C₁-C₈-alkylsulphanyl; C₁-C₆-halogenoalkylsulphanyl comprising up to 9 halogen atoms that can be the same or different; C₂-C₈-alkenyloxy; C₂-C₈-halogenoalkenyloxy comprising up to 9 halogen atoms that can be the same or different; C₃-C₈-alkynyloxy; C₃-C₈-halogenoalkynyloxy comprising up to 9 halogen atoms that can be the same or different; C₃-C₈-cycloalkyl; C₁-C₈-alkoxycarbonyl; C₁-C₈-alkylsulphinyl; C₁-C₈-alkylsulphonyl; C₁-C₈-halogenoalkylsulphinyl comprising up to 9 halogen atoms that can be the same or different; C₁-C₈-halogenoalkylsulphonyl comprising up to 9 halogen atoms that can be the same or different or C₁-C₆-alkoximino-C₁-C₆-alkyl;
n represents 0, 1, 2 or 3;
R^{a} and R^{b}, that can be the same or different, represent a hydrogen atom; C₁-C₈-alkyl; C₁-C₈-halogenoalkyl comprising up to 9 halogen atoms that can be the same or different; C₁-C₈-alkoxy-C₁-C₈-alkyl; C₂-C₈-alkenyl; C₂-C₈-halogenoalkenyl comprising up to 9 halogen atoms that can be the same or different; C₂-C₈-alkynyl; C₂-C₈-halogenoalkynyl comprising up to 9 halogen atoms that can be the same or different; C₃-C₇-cycloalkyl; C₃-C₇-cycloalkyl-C₁-C₈-alkyl; C₃-C₇-halogenocycloalkyl comprising up to 9 halogen atoms that can be the same or different; formyl; C₁-C₈-alkylcarbonyl; C₁-C₈-halogenoalkylcarbonyl comprising up to 9 halogen atoms that can be the same or different; C₁-C₈-alkylsulphonyl; C₁-C₈-halogenoalkylsulphonyl comprising up to 9 halogen atoms that can be the same or different; phenyl that can be substituted by up to 5 groups Q; naphthyl that can be substituted by up to 6 groups Q; phenylmethylene that can be substituted by up to 5 groups Q; phenylsulphonyl that can be substituted by up to 5 groups Q;
Q, that can be the same or different, represents a halogen atom; cyano; nitro; C₁-C₈-alkyl; C₁-C₈-alkoxy; C₁-C₈-alkylsulfanyl; C₁-C₈-halogenoalkyl comprising up to 9 halogen atoms that can be the same or different; C₁-C₈-halogenoalkoxy comprising up to 9 halogen atoms that can be the same or different;
as well as salts, N-oxides, metallic complexes, metalloidic complexes and optically active or geometric isomers thereof.

2. A compound according to claim 1 wherein A is selected in the list consisting of:
a heterocycle of formula (A¹) 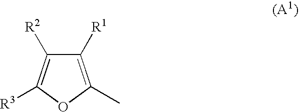
wherein R¹ to R³ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²) 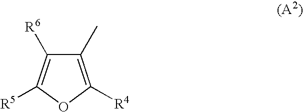
wherein R⁴ to R⁶ that can be the same or different represent a hydrogen atom; a halogen atom; C₁-C₅-alkyl or C₁-C₅-halogenoalkyl comprising up to 5 halogen atoms that can be the same or different;
a heterocycle of formula (A³) 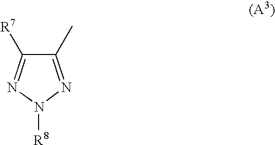
wherein R⁷ represents a hydrogen atom; a halogen atom; C₁-C₅-alkyl or C₁-C₅-halogenoalkyl comprising up to 5 halogen atoms that can be the same or different;
R⁸ represents a hydrogen atom; C₁-C₅-alkyl or a phenyl substituted by a halogen atom or by a C₁-C₅-alkyl;
a heterocycle of formula (A⁴) 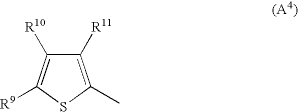
wherein R⁹ to R¹¹ that can be the same or different represent a hydrogen atom; a halogen atom; C₁-C₅-alkyl; an amino; C₁-C₅-alkoxy; C₁-C₅-alkylsulphanyl or C₁-C₅-halogenoalkyl comprising up to 5 halogen atoms that can be the same or different;
a heterocycle of formula (A⁵) 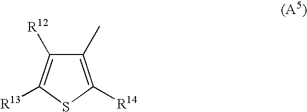
wherein
R¹² and R¹³ that can be the same or different represent a hydrogen atom; a halogen atom; C₁-C₅-alkyl; C₁-C₅-alkoxy; an amino or C₁-C₅-halogenoalkyl comprising up to 5 halogen atoms that can be the same or different;
R¹⁴ represents a hydrogen atom; a halogen atom; C₁-C₅-alkyl; an amino or C₁-C₅-halogenoalkyl comprising up to 5 halogen atoms that can be the same or different;
a heterocycle of formula (A⁶) 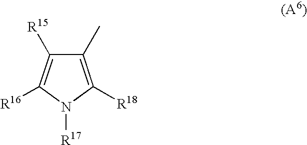
wherein:
R¹⁵ represents a hydrogen atom; a halogen atom; a cyano; C₁-C₅-alkyl or C₁-C₅-halogenoalkyl comprising up to 5 halogen atoms that can be the same or different;
R¹⁶ and R¹⁸ that can be the same or different represent a hydrogen atom; a halogen atom; C₁-C₅-alkoxycarbonyl; C₁-C₅-alkyl or C₁-C₅-halogenoalkyl comprising up to 5 halogen atoms that can be the same or different;
R¹⁷ represent a hydrogen atom or C₁-C₅-alkyl; C₁-C₅-halogenoalkyl comprising up to 5 halogen atoms that can be the same or different or a phenyl substituted by a halogen atom or by a C₁-C₅-alkyl;
a heterocycle of formula (A⁷) 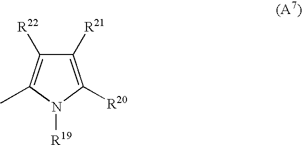
wherein:
R¹⁹ represents a hydrogen atom; C₁-C₅-alkyl or a phenyl substituted by a halogen atom or by a C₁-C₅-alkyl;
R²⁰ to R²² that can be the same or different represent a hydrogen atom; a halogen atom; C₁-C₅-alkyl or C₁-C₅-halogenoalkyl comprising up to 5 halogen atoms that can be the same or different;
a heterocycle of formula (A⁸) 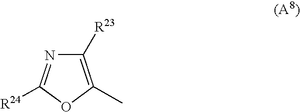
wherein:
R²³ represents a hydrogen atom; a halogen atom; C₁-C₅-alkyl; C₁-C₅-halogenoalkyl comprising up to 5 halogen atoms that can be the same or different;
R²⁴ represents a hydrogen atom or C₁-C₅-alkyl;
a heterocycle of formula (A⁹) 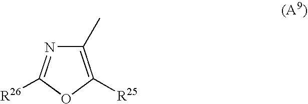
wherein:
R²⁵ represents a halogen atom; C₁-C₅-alkyl or C₁-C₅-halogenoalkyl comprising up to 5 halogen atoms that can be the same or different;
R²⁶ represents a hydrogen atom or C₁-C₅-alkyl;
a heterocycle of formula (A¹⁰) 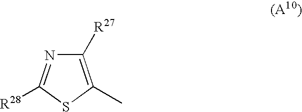
wherein:
R²⁷ represents a halogen atom; C₁-C₅-alkyl or C₁-C₅-halogenoalkyl comprising up to 5 halogen atoms that can be the same or different;
R²⁸ represents a hydrogen atom; a halogen atom; an amino; C₁-C₅-alkyl; C₁-C₅-halogenoalkyl comprising up to 5 halogen atoms that can be the same or different or a phenyl substituted by a halogen atom or by a C₁-C₅-alkyl;
a heterocycle of formula (A¹¹) 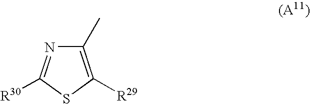
wherein:
R²⁹ represents a hydrogen atom; a halogen atom; C₁-C₅-alkyl or C₁-C₅-halogenoalkyl comprising up to 5 halogen atoms that can be the same or different;
R³⁰ represents a hydrogen atom; a halogen atom; C₁-C₅-alkyl; C₁-C₅-halogenoalkyl comprising up to 5 halogen atoms that can be the same or different; an amino; C₁-C₅-alkylamino; di-C₁ C₅-alkylamino;
a heterocycle of formula (A¹²) 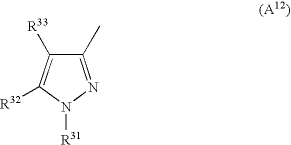
wherein:
R³¹ represents a hydrogen atom; a halogen atom; C₁-C₅-alkyl or a phenyl substituted by a halogen atom or by a C₁-C₅-alkyl;
R³² represents a hydrogen atom; a halogen atom; C₁-C₅-alkyl or C₁-C₅-halogenoalkyl comprising up to 5 halogen atoms that can be the same or different;
R³³ represents a hydrogen atom; a halogen atom; a nitro or C₁-C₅-halogenoalkyl comprising up to 5 halogen atoms that can be the same or different;
a heterocycle of formula (A¹³) 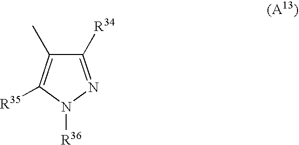
wherein:
R³⁴ represents a hydrogen atom; a halogen atom; C₁-C₅-alkyl; C₃-C₅-cycloalkyl; C₁-C₅-halogenoalkyl comprising up to 5 halogen atoms that can be the same or different; C₁-C₅-alkoxy; C₂-C₅-alkynyloxy; C₁-C₅-halogenoalkoxy comprising up to 5 halogen atoms that can be the same or different or a phenyl substituted by a halogen atom or by a C₁-C₅-alkyl;
R³⁵ represents a hydrogen atom; a halogen atom; C₁-C₅-alkyl; a cyano; C₁-C₅-alkoxy;
C₁-C₅-alkylsulphanyl; C₁-C₅-halogenoalkyl comprising up to 5 halogen atoms that can be the same or different; C₁-C₅-halogenoalkoxy comprising up to 5 halogen atoms that can be the same or different; an amino; C₁-C₅-alkylamino or di(C₁-C₅-alkyl)-amino;
R³⁶ represents a hydrogen atom; C₁-C₅-alkyl or a phenyl substituted by a halogen atom or by a C₁-C₅-alkyl;
a heterocycle of formula (A¹⁴) 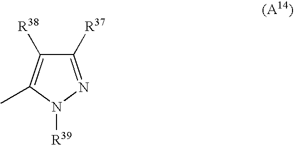
wherein:
R³⁷ and R³⁸ that can be the same or different represent a hydrogen atom; a halogen atom; C₁-C₅-alkyl; C₁-C₅-halogenoalkyl comprising up to 5 halogen atoms that can be the same or different; C₁-C₅-alkoxy or a C₁-C₅-alkylsulphanyl;
R³⁹ represents a hydrogen atom; C₁-C₅-alkyl or a phenyl substituted by a halogen atom or by a C₁-C₅-alkyl;
a heterocycle of formula (A¹⁵) 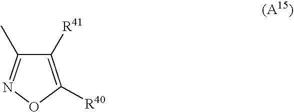
wherein R⁴⁰ and R⁴¹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A¹⁶) 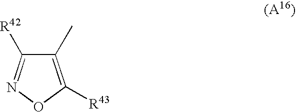
wherein R⁴² and R⁴³ that can be the same or different represent a hydrogen atom; C₁-C₅-alkyl; C₁-C₅-halogenoalkyl comprising up to 5 halogen atoms that can be the same or different; an amino or a phenyl substituted by a halogen atom or by a C₁-C₅-alkyl;
a heterocycle of formula (A¹⁷) 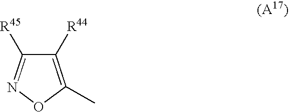
wherein R⁴⁴ and R⁴⁵ that can be the same or different represent a hydrogen atom; a halogen atom; C₁-C₅-alkyl or C₁-C₅-halogenoalkyl comprising up to 5 halogen atoms that can be the same or different;
a heterocycle of formula (A¹⁸) 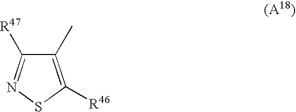
wherein:
R⁴⁶ represents a hydrogen atom; C₁-C₅-alkyl; C₁-C₅-halogenoalkyl comprising up to 5 halogen atoms that can be the same or different or C₁-C₅-alkylsulfanyl;
R⁴⁷ represents a hydrogen atom; a halogen atom or C₁-C₅-alkyl;
a heterocycle of formula (A¹⁹) 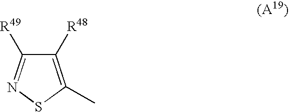
wherein R⁴⁸ and R⁴⁹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²⁰) 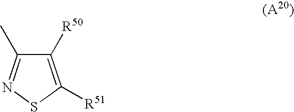
wherein R⁵⁰ and R⁵¹ that can be the same or different represent a hydrogen atom; a halogen atom; C₁-C₅-alkyl or C₁-C₅-halogenoalkyl comprising up to 5 halogen atoms that can be the same or different;
a heterocycle of formula (A²¹) 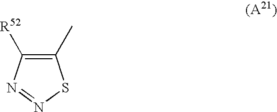
wherein R⁵² represents a halogen atom; C₁-C₅-alkyl or C₁-C₅-halogenoalkyl comprising up to 5 halogen atoms that can be the same or different;
a heterocycle of formula (A²²) 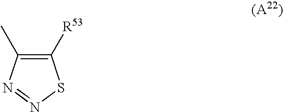
wherein R⁵³ represents a halogen atom; C₁-C₅-alkyl or C₁-C₅-halogenoalkyl comprising up to 5 halogen atoms that can be the same or different;
a heterocycle of formula (A²³) 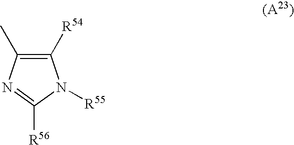
wherein:
R⁵⁴ and R⁵⁶ that can be the same or different represent a hydrogen atom; a halogen atom; C₁-C₅-alkyl or C₁-C₅-halogenoalkyl comprising up to 5 halogen atoms that can be the same or different;
R⁵⁵ represents a hydrogen atom or C₁-C₅-alkyl;
a heterocycle of formula (A²⁴) 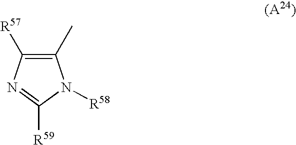
wherein:
R⁵⁷ and R⁵⁹ that can be the same or different represent a hydrogen atom; a halogen atom; C₁-C₅-alkyl or C₁-C₅-halogenoalkyl comprising up to 5 halogen atoms that can be the same or different;
R⁵⁸ represents a hydrogen atom or C₁-C₅-alkyl;
a heterocycle of formula (A²⁵) 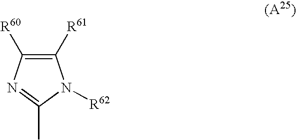
wherein:
R⁶⁰ and R⁶¹ that can be the same or different represent a hydrogen atom; a halogen atom; C₁-C₅-alkyl or C₁-C₅-halogenoalkyl comprising up to 5 halogen atoms that can be the same or different;
R⁶² represents a hydrogen atom or C₁-C₅-alkyl;
a heterocycle of formula (A²⁶) 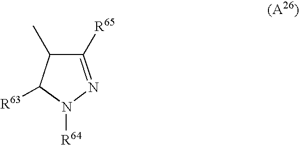
wherein:
R⁶⁵ represents a hydrogen atom; a halogen atom; C₁-C₅-alkyl; C₃-C₅-cycloalkyl; C₁-C₅-halogenoalkyl comprising up to 5 halogen atoms that can be the same or different; C₁-C₅-alkoxy; C₂-C₅-alkynyloxy or C₁-C₅-halogenoalkoxy comprising up to 5 halogen atoms that can be the same or different;
R⁶³ represents a hydrogen atom; a halogen atom; C₁-C₅-alkyl; a cyano; C₁-C₅-alkoxy; C₁-C₅-alkylsulphanyl; C₁-Cₛ-halogenoalkyl comprising up to 5 halogen atoms that can be the same or different; C₁-C₅-halogenoalkoxy comprising up to 5 halogen atoms that can be the same or different; amino; C₁-C₅-alkylamino or di(C₁-C₅-alkyl)amino;
R⁶⁴ represents a hydrogen atom or C₁-C₅-alkyl.

3. A compound according to claim 1 wherein A represents a 5-membered heterocycle that is substituted in position ortho or wherein A represents an α-substituted 5-membered heterocycle.

4. A compound according to claim 1 wherein A represents A¹³.

5. A compound according to claim 1 wherein T represents sulphur.

6. A compound according to claim 1 wherein Z¹ represents a non-substituted C₃-C₇-cycloalkyl.

7. A compound according to claim 1 wherein Z¹ represents cyclopropyl.

8. A compound according to claim 1 wherein Z² and Z³, that can be the same or different, represent a hydrogen atom or C₁-C₅-alkyl.

9. A compound according to claim 1 wherein Y represents trifluoromethyl.

10. A compound according to claim 1 wherein X, that can be the same or different, represents a halogen atom; C₁-C₈-alkyl; C₁-C₆-halogenoalkyl comprising up to 5 halogen atoms that can be the same or different; C₁ C₈-alkoxy; C₁-C₆-halogenoalkoxy comprising up to 5 halogen atoms that can be the same or different.

11. A compound according to claim 1 wherein n represents 1.

12. A fungicide composition comprising, as an active ingredient, an effective amount of a compound of formula (I) according to claim 1 and an agriculturally acceptable support, carrier or filler.

13. A method for controlling phytopathogenic fungi of crops, characterized in that an agronomically effective and substantially non-phytotoxic quantity of a compound according to claim 1 is applied to the soil where plants grow or are capable of growing, to the leaves and/or the fruit of plants or to the seeds of plants.

14. A method for controlling phytopathogenic fungi of crops, characterized in that an agronomically effective and substantially non-phytotoxic quantity of a composition according to claim 12 is applied to the soil where plants grow or are capable of growing, to the leaves and/or the fruit of plants or to the seeds of plants.
